(19) United States
(12) Patent Application Publication
(10) Pub. No.: US 2014/0051597 A1
(43) Pub. Date: Feb. 20, 2014

(54) Antibody Biomarkers for Diabetes

(71) Applicants: Minnie M. Sarwal, Portola Valley, CA (US); Tara Sigdel, Palo Alto, CA (US)

(72) Inventors: Minnie M. Sarwal, Portola Valley, CA (US); Tara Sigdel, Palo Alto, CA (US)

(73) Assignee: The Board of Trustees of the Leland Stanford Junio University, Palo Alto (CA)

(21) Appl. No.: 14/001,825

(22) PCT Filed: Apr. 6, 2012
(86) PCT No.: PCT/US2012/032610

§ 371 Date: Nov. 4, 2013

Related U.S. Application Data
(60) Provisional application No. 61/472,411, filed on Apr. 6, 2011.

(51) Int. Cl. G01N 33/68

(52) U.S. Cl. CPC G01N 33/6854
USPC 506/9; 436/501; 435/7.92; 506/18

(57) ABSTRACT

Methods are provided for determining whether a subject has a diabetes phenotype. In practicing the subject methods, a sample, e.g., a blood sample, from a subject is analyzed for the presence of one or more autoantibodies to obtain an antibody signature. The obtained antibody signature is then employed to determine whether the subject has a diabetes phenotype. The subject methods may be used in diagnostic or prognostic applications, e.g., determining whether the subject has diabetes (e.g., T1D or T2D), or monitoring a subject with diabetes to determine whether the subject has or will develop ESRD. Also provided are compositions, systems and kits that find use in practicing the subject methods. The subject methods and compositions find use in a variety of applications, including the diagnosis and monitoring of diabetes in a subject.

### CROSS-REFERENCE TO RELATED APPLICATIONS

Pursuant to 35 U.S.C. §119 (e), this application claims priority to the filing date of U.S. Provisional Patent Application Ser. No. 61/472,411 filed on Apr. 6, 2011, the disclosure of which application is herein incorporated by reference.

### INTRODUCTION

Diabetes is a chronic disease marked by high levels of sugar in the blood due to a lack of insulin produced in the pancreas. Two major types of diabetes include Type I diabetes and Type 2 diabetes.

Type 1 diabetes (T1D) results from autoimmune destruction of insulin-producing beta cells of the pancreas. Symptoms include polyuria (frequent urination), polydipsia (increased thirst), polyphagia (increased hunger), and weight loss. T1D is fatal unless treated with insulin and must be continued indefinitely, although many people who develop the disease are otherwise healthy and treatment need not significantly impair normal activities. Type 2 diabetes (T2D) is becoming more common due to increasing obesity and failure to exercise. In T2D the pancreas does not make enough insulin to keep blood glucose levels normal, in some cases because the body does not respond well to insulin. Many subjects with T2D do not know they have it, although it is a serious condition.

Treatment for diabetes is burdensome for many people. Complications may be associated with both low blood sugar and high blood sugar. Low blood sugar may lead to seizures or episodes of unconsciousness and may require emergency treatment. High blood sugar may lead to increased fatigue and can also result in long term damage to organs.

Diabetes is the leading cause of end-stage renal disease (ESRD) (i.e., kidney failure requiring dialysis or transplantation). The basic function of the kidneys is to filter waste products from the blood. If blood glucose levels are too high, the kidneys must work harder to maintain the necessary filtering processes. The extra force required may cause the capillaries in the kidneys to leak, allowing protein to be lost in the urine. Eventually, the kidneys lose their ability to function, and waste products will build up in the blood, requiring dialysis or a transplant. Diabetic kidney disease or diabetic nephropathy can lead to ESRD. Early detection of kidney problems is of interest as in its earlier stages, diabetic kidney disease may go unnoticed. Persistent elevated microalbumin levels seen in people with T1D suggest they are in the earliest stages of kidney disease. Similar levels seen in people with T2D should be a considered a warning that diabetic kidney disease is developing.

As such, diagnostic and prognostic tests that find use in the diagnosis and monitoring of diabetes in a subject are of interest.

### SUMMARY

Methods for determining whether a subject has a diabetes phenotype are provided. Aspects of the invention include analyzing a sample for the presence of one or more autoantibodies to obtain an antibody signature. The obtained antibody signature is then employed to determine whether the subject has a diabetes phenotype. Embodiments of the methods may be used in diagnostic or prognostic applications, e.g., determining whether the subject has diabetes (e.g., T1D or T2D), or monitoring a subject with diabetes to determine whether the subject has or will develop ESRD. Also provided are compositions, systems and kits that find use in practicing the subject methods. The subject methods and compositions find use in a variety of applications, including the diagnosis and monitoring of diabetes in a subject.

Accordingly, aspects of the subject invention provide methods for diagnosing or monitoring diabetes in a subject. In certain embodiments, the methods include obtaining a sample from the subject (e.g., a blood or serum sample) and determining the level of one or more autoantibodies therein to obtain an antibody signature of the sample. The antibody signature can then be used to determine the diabetes phenotype of the subject, e.g., by comparing to one or more antibody signatures from subjects known to not have diabetes. Such known antibody signatures can also be called controls or reference signatures/profiles.

Aspects of the invention include methods of diagnosing diabetes in a subject. Other aspects of the invention include methods of monitoring diabetes in a subject over time by determining and following changes in the antibody signatures of samples of the subject over time.

In certain embodiments the method includes: (a) evaluating the level of one or more autoantibodies in a sample from a subject to obtain an antibody signature; and (b) determining the diabetes phenotype of the subject based on the antibody signature. In certain embodiments, the antibody signature comprises autoantibody level data for one or more autoantibodies specific for proteins of Tables 1-4 (see below).

### Definitions

For convenience, certain terms employed in the specification, examples, and appended claims are collected here.

The term “autoantibody” as used herein refers to an antibody produced by an individual, where the antibody is directed against one or more ‘self’ antigens (e.g., antigens that are native to the individual, e.g., an antigen on a cell or tissue, or an endogenous peptide or protein).

The term “antibody signature” as used herein refers to the level of one or more antibodies, e.g., autoantibodies, in a sample. The level of an antibody in a sample (e.g., an autoantibody) may be qualitative or quantitative in nature. The term “diabetes phenotype” as used herein refers to an observable characteristic or trait of a subject who has diabetes (e.g., T1D or T2D), or has diabetic kidney disease (e.g., ESRD or an early stage thereof). For example, a diabetes phenotype may include increased levels of one or more autoantibodies in the subject that is experiencing diabetes as compared to a healthy subject. For example, a diabetes phenotype may include increased levels of one or more autoantibodies in a subject with diabetes that is experiencing ESRD as compared to a control subject. In some cases, the diabetes phenotype may include decreased levels of one or more autoantibodies in the subject as compared to a control subject.

The terms “reference” and “control” are used interchangeably to refer to a known value or set of known values against which an observed value may be compared. As used herein, known means that the value represents an understood parameter, e.g., a level of an autoantibody in a diabetes phenotype. A reference or control value may be from a single measurement or data point or may be a value calculated based on more than one measurement or data point (e.g., an average of many different measurements). Any convenient reference or control value(s) may be employed in practicing aspects of the subject invention.

The terms “protein”, “polypeptide”, “peptide” and the like refer to a polymer of amino acids (an amino acid sequence) and does not refer to a specific length of the molecule. This term also refers to or includes any modifications of the polypeptide (e.g., post-translational), such as glycosylations, acetylations, phosphorylations and the like. Included within the definition are, for example, polypeptides containing one or more analogs of an amino acid, polypeptides with substituted linkages, as well as other modifications known in the art, both naturally occurring and non-naturally occurring.

The terms “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” may include determining the amount of something present, as well as determining whether it is present or absent. In some instances, the term “determining” is used in connection with the evaluation of whether a subject has a condition of interest, e.g., a disease condition. In other words, the term determining may be used interchangeably with diagnosing. In such instances, the determination that is made is an ascertainment that the subject has the condition of interest based on data obtained as described herein, where the subject may or may not in fact have the condition of interest. Accordingly, methods of invention include methods which are not 100% accurate. Even though such determinations are not 100% accurate, they still provide useful information, e.g., in the context of making a decision that a subject is more likely than not to have a condition, is sufficiently likely to have a condition such that further a further evaluation (e.g., in the form of a second diagnostic test) or treatment regimen is warranted, etc.

The terms “profile” and “signature” and “result” and “data”, and the like, when used to describe antibody/protein/peptide level or gene expression level data are used interchangeably (e.g., antibody signature/profile/result/data, gene expression signature/profile/result/data, etc.).

### DETAILED DESCRIPTION

Methods for determining whether a subject has a diabetes phenotype are provided. Aspects of the invention include analyzing a sample for the presence of one or more autoantibodies to obtain an antibody signature. The obtained antibody signature is then employed to determine whether the subject has a diabetes phenotype. Embodiments of the methods may be used in diagnostic or prognostic applications, e.g., determining whether the subject has diabetes (e.g., T1D or T2D), or monitoring a subject with diabetes to determine whether the subject has or will develop ESRD. Also provided are compositions, systems and kits that find use in practicing the subject methods. The subject methods and compositions find use in a variety of applications, including the diagnosis and monitoring of diabetes in a subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the invention include methods of determining whether a subject has a diabetes phenotype, as well as reagents and kits for use in practicing the subject methods. In further describing aspects of the invention, embodiments of the methods are described first in greater detail, followed by a review of embodiments of reagents and kits that find use in practicing the methods described herein.

### Methods of Diagnosing and Monitoring Diabetes

The embodiments described herein provide methods of determining whether a patient or subject has a diabetes phenotype. By diabetes phenotype is meant an observable characteristic or trait of a subject who has diabetes (e.g., T1D or T2D), or a diabetic kidney disease (e.g., ESRD or the early stages thereof).

In some embodiments, the subject methods include determining whether individuals have a diabetes phenotype. In certain embodiments, the method can be considered a method of diagnosing diabetes (e.g., T1D or T2D) in a subject. In certain embodiments, the method can be considered a method of monitoring diabetes in a subject to determine whether the subject has a diabetic kidney disease (e.g., ESRD or the early stages thereof). A diabetes phenotype may indicate that an individual has either T1D or T2D, or that an individual with diabetes has or will develop ESRD in the future, and as such may be used in diagnostic or prognostic applications.

In practicing the subject methods, a subject or patient sample (e.g., a blood sample, a urine sample, cells or a tissue sample) is assayed to determine whether the host from which the assayed sample was obtained has a diabetes phenotype. Accordingly, the subject methods include obtaining a suitable sample from the subject or patient of interest. The sample is derived from any initial suitable source, where sample sources of interest include, but are not limited to, many different physiological sources, e.g., CSF, urine, saliva, tears, tissue derived samples, e.g., homogenates, and blood or derivatives thereof.

In certain embodiments, a suitable initial source for the patient sample is blood. As such, the sample employed in the subject assays of these embodiments may be a blood-derived sample. The blood derived sample may be derived from whole blood or a fraction thereof, e.g., serum, plasma, etc., where in some embodiments the sample is derived from blood cells harvested from whole blood. Of particular interest as a sample source are peripheral blood mononuclear cells (PBMCs), e.g., peripheral blood lymphocytes (PBL).

Any suitable protocol for obtaining such samples may be employed. Moreover, in certain embodiments, samples may be obtained from a third party (e.g., a sample may be obtained from a third party that independently collects the sample from a subject).

In practicing the subject methods, the sample (e.g., blood or urine sample) is assayed to obtain an antibody signature of the sample, or protein profile, in which the amount of one or more specific autoantibodies to peptides/proteins in the sample is determined, where the determined amount may be relative and/or quantitative in nature. As such, in certain embodiments the level of only one autoantibody is evaluated. In yet other embodiments, the levels of two or more, e.g., about 3 or more, about 4 or more, about 5 or more, about 6 or more, about 7 or more, about 8 or more, about 9 or more, about 10 or more, about 15 or more, about 20 or more, about 25 or more, about 30 or more, about 40 or more, about 50 or more, about 100 or more, about 200 or more, etc., autoantibodies is evaluated. Accordingly, in the subject methods, the level of one or more autoantibodies in a sample is evaluated.

In many embodiments, a sample is assayed to generate an antibody profile (or signature) that includes level data for one or more autoantibody, and in some cases a plurality of autoantibodies, where by plurality is meant two or more different autoantibodies, such as about 5 or more, about 10 or more, about 20 or more different autoantibodies or more, such as 50 or more, 100 or more, etc. In certain embodiments, the antibody signature includes measurements for the amount of one or more autoantibodies to proteins (or peptides derived therefrom) shown in Tables 1-4.

In the broadest sense, the evaluation of autoantibody levels may be qualitative or quantitative. As such, where detection is qualitative, the methods provide a reading or evaluation, e.g., assessment, of whether or not the target analyte (e.g., autoantibody), is present in the sample being assayed. In yet other embodiment, the methods provide a quantitative detection of whether the target analyte is present in the sample being assayed, i.e., an evaluation or assessment of the actual amount or relative abundance of the target analyte, e.g., autoantibody in the sample being assayed. In such embodiments, the quantitative detection may be absolute or, if the method is a method of detecting two or more different analytes in a sample, relative. As such, the term “quantifying” when used in the context of quantifying a target analyte, e.g., antibody, in a sample can refer to absolute or to relative quantification. Absolute quantification may be accomplished by inclusion of known concentration(s) of one or more control analytes and referencing the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other.

In some embodiments, the invention provides methods for determining whether a patient or subject has a diabetes phenotype with a high positive predictive value (PPV). The term “PPV” is used in its art accepted manner and defined as True Positives (TP)/(TP+False Positives (FP)), In some instances, the determination that is made has a PPV that 60, 70, 80, 90, 95, or 99.9% or higher. In some embodiments, the invention provides methods for determining whether a patient or subject has a diabetes phenotype, wherein the PPV is equal or higher than 80%. In some embodiments, the invention provides methods for determining whether a patient or subject has a diabetes phenotype, wherein the negative predictive value (NPV) is 60, 70, 80, 90, 95, or 99.9% or higher. The term “NPV” is used in its art accepted manner and is defined as True Negatives (TN)/(TN+False Negatives (FN)). In some embodiments, the invention provides methods for determining whether a patient or subject has a diabetes phenotype, wherein the NPV is higher than 80%.

In some embodiments, the invention provides methods for determining whether a patient or subject has a diabetes phenotype with a high specificity. The term “specificity” is used in its art accepted manner and is defined as TN/(TN+FP). In some instances, the specificity is 60, 70, 80, 90, 95, or 99.9% or higher. In some embodiments, the invention provides methods for determining whether a patient or subject has a diabetes phenotype, wherein the specificity is equal or higher than 80%.

In some embodiments, the invention provides methods for determining whether a patient or subject has a diabetes phenotype with a high sensitivity. The term “sensitivity” is used in its art accepted manner and is defined as TP/(TP+FN). In some instances, the sensitivity of the methods is 60, 70, 80, 90, 95, or 99.9 or higher. In some embodiments, the invention provides methods for determining whether a patient or subject has a diabetes phenotype wherein the sensitivity is higher than 80%.

In some embodiments, the invention provides methods for determining whether a patient or subject has a diabetes phenotype, wherein the Area Under the Curve (AUC) value is 0.5, 0.6, 07, 0.8 or 0.9 or higher. The term “AUC” is used in its art accepted manner and is defined as the area under the Receiver Operating Characteristic (ROC) curve. The ROC curve is used in its art accepted manner and is defined as a plot of test sensitivity (True Positive Rate: TPR) versus (1-specificity) (False Positive Rate: FPR). In some embodiments, the invention provides methods for determining whether a patient or subject has a diabetes phenotype, wherein the AUC value is 0.7 or higher. In some embodiments, the invention provides methods for determining whether a patient or subject has a diabetes phenotype, wherein the AUC value is 0.8 or higher. In some embodiments, the invention provides methods for determining whether a patient or subject has a diabetes phenotype, wherein the AUC value is 0.9 or higher.

In some embodiments, the p value in the analysis of the methods described herein is 0.05, 04, 0.03, 0.02, 0.01, 0.009, 0.005, or 0.001 or below. In some embodiments, the p value is 0.001 or below. Thus in some embodiments, the invention provides methods for determining whether a patient or subject has a diabetes phenotype, wherein the p value is 0.05, 04, 0.03, 0.02, 0.01, 0.009, 0.005, or 0.001 or below. In some embodiments, the p value is 0.001 or below.

In certain embodiments, autoantibodies of interest are autoantibodies that are present at different levels in individuals with diabetes (e.g., T1D or T2D) and/or ESRD or the early stages thereof versus healthy individuals. Representative autoantibodies of interest in these embodiments include, but are not limited to, the autoantibodies to proteins provided in Tables 1-4, where the Entrez Gene symbol for each protein is listed. (Note that detailed information for each protein in Tables 1-4, including sequence information, can be retrieved through the NCBI Entrez database located at the website http (colon)//www (dot) ncbi.nlm.nih(dot)gov). As such, the antibody signature may contain include autoantibody level data for one autoantibody, 2 or more autoantibodies, 3 or more autoantibodies, 4 or more autoantibodies, 5 or more autoantibodies, 6 or more autoantibodies, 7 or more autoantibodies, 8 or more autoantibodies, 9 or more autoantibodies, 10 or more autoantibodies, 12 or more autoantibodies, 14 or more autoantibodies, 16 or more autoantibodies, 18 or more autoantibodies, etc., specific for proteins that are described herein.

In certain embodiments, at least one of the autoantibodies in the antibody profile is from autoantibodies to proteins listed Table 1, where the antibody profile may include level data for any combination of the autoantibodies to proteins listed in Table 1 (e.g., 1, 2, 3, 4, 5, 6, 7, 8, 9, 10, 12, 14, 16, 18, 20, 25 or 30 autoantibodies to proteins listed in Table 1). In certain embodiments, at least one of the autoantibodies in the antibody profile is from autoantibodies to proteins listed Table 2, where the antibody profile may include level data for any combination of the autoantibodies to proteins listed in Table 2 (e.g., 1, 2, 3, 4, 5, 6, 7, 8, 9, 10, 12, 14, 16, 18, 20, 25 or 30 autoantibodies to proteins listed in Table 2). In certain embodiments, at least one of the autoantibodies in the antibody profile is from autoantibodies to proteins listed Table 3, where the antibody profile may include level data for any combination of the autoantibodies to proteins listed in Table 3 (e.g., 1, 2, 3, 4, 5, 6, 7, 8, 9, 10, 12, 14, 16, 18, 20, 25 or 29 autoantibodies to proteins listed in Table 3). In certain embodiments, at least one of the autoantibodies in the antibody profile is from autoantibodies to proteins listed Table 4, where the antibody profile may include level data for any combination of the autoantibodies to proteins listed in Table 4 (e.g., 1, 2, 3, 4, 5, 6, 7, 8, 9, 10, 12, 14, 16, 18, 20, 25 or 30 autoantibodies to proteins listed in Table 4).

Tables 1 to 4 show lists of autoantibodies to proteins whose presence and level in a sample can be used to determine a particular diabetes phenotype in a subject. In some embodiments, the levels of these autoantibodies is significantly different in a diabetes phenotype as compared to a non-diabetes phenotype (e.g., autoantibodies are significantly higher, or alternatively, significantly lower in a subject with diabetes as compared to a normal control).

In certain embodiments, one or more autoantibodies to proteins of Table 1 can be used to determine a diabetes phenotype in a subject with T1D as compared to a healthy subject. In certain embodiments, the one or more autoantibodies include at least one antibody specific for a protein selected from ACY3, AMMECR1L, BATF2, BMX, EPHA2, FLT1, PAK4, TRAF3IP2, C9ORF25, CXORF38 and CXORF56. In certain embodiments, the one or more autoantibodies in the antibody signature includes autoantibodies specific for the proteins ACY3, AMMECR1L, BATF2 and BMX. In such embodiments, the subject is determined to have a diabetes phenotype when the level of autoantibodies specific for one or more of these proteins in the sample is increased as compared to a control reference antibody signature. In certain embodiments, the one or more autoantibodies in the antibody signature include an autoantibody specific for the protein ACY3. In such embodiments, the subject is determined to have a diabetes phenotype when the level of the autoantibody to ACY3 in the sample is increased as compared to a control reference antibody signature.

In certain embodiments, one or more autoantibodies to proteins of Table 3 can be used to determine a diabetes phenotype in a subject with T2D as compared to a healthy subject. In certain embodiments, the one or more autoantibodies include at least one antibody specific for a protein selected from NADK, MED9, LDHA, ARHGAP26, ANKRA2, CRY2, IL23A, DUSP14, ZBTB44, SIRT1 and SLC2A3. In certain embodiments, the one or more autoantibodies in the antibody signature includes autoantibodies specific for the proteins NADK, MED9, LDHA and ARHGAP26. In such embodiments, the subject is determined to have a diabetes phenotype when the level of autoantibodies specific for one or more of these proteins in the sample is increased as compared to a control reference antibody signature. In certain embodiments, the one or more autoantibodies in the antibody signature include an autoantibody specific for the protein NADK. In such embodiments, the subject is determined to have a diabetes phenotype when the level of the autoantibody to NADK in the sample is increased as compared to a control reference antibody signature.

In certain embodiments, one or more autoantibodies to proteins of Table 4 can be used to determine a diabetes phenotype in a subject with ESRD as compared to a control subject. In certain embodiments, the one or more autoantibodies include at least one antibody specific for a protein selected from IGLC1, IGHG1, EDC3, APEX2, CD3D, TRIM21, IGKV1-5, IGHG3, CTLA-FC, CD7 and CLIP4. In certain embodiments, the one or more autoantibodies in the antibody signature includes autoantibodies specific for the proteins IGLC1, IGHG1, EDC3 and APEX2. In such embodiments, the subject is determined to have a diabetes phenotype when the level of autoantibodies specific for one or more of these proteins in the sample is increased as compared to a control reference antibody signature. In certain embodiments, the one or more autoantibodies in the antibody signature include an autoantibody specific for the protein IGLC1. In such embodiments, the subject is determined to have a diabetes phenotype when the level of the autoantibody to IGLC1 in the sample is increased as compared to a control reference antibody signature.

In certain embodiments, one or more autoantibodies to proteins of Table 2 can be used to determine a diabetes phenotype in a subject with T2D who has not developed ESRD, as compared to a control subject. In certain embodiments, the one or more autoantibodies include at least one antibody specific for a protein selected from RAD51AP1, HADH, C11orf16, TAC3, ABR, ECE1, PPP1 R2, GRINL1A, C19orf44, MUSTN1 and ETHE1. In certain embodiments, the one or more autoantibodies in the antibody signature includes autoantibodies specific for the proteins RAD51 AP1, HADH, C11orf16 and TAC3. In such embodiments, the subject is determined to have a diabetes phenotype when the level of autoantibodies specific for one or more of these proteins in the sample is increased as compared to an antibody signature from a subject with ESRD. In certain embodiments, the one or more autoantibodies in the antibody signature include an autoantibody specific for the protein RAD51AP1. In such embodiments, the subject is determined to have a diabetes phenotype when the level of the autoantibody to RAD51AP1 in the sample is increased as compared to an antibody signature from a subject with ESRD.

The selection of which autoantibodies to proteins from Tables 1-4 that are to be included in the antibody signature will be determined by the desires of the user. No limitation in this regard is intended. It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to autoantibodies to proteins that find use as markers for diagnosing or monitoring diabetes are specifically embraced by the present invention and are disclosed herein just as if each and every combination was individually and explicitly disclosed. As such, any combination of autoantibodies to proteins from Tables 1- 4 are disclosed herein just as if each and every such sub-combination of proteins was individually and explicitly disclosed herein.

In certain embodiments, additional analytes beyond those listed above may be assayed, where the additional analytes may be additional proteins (e.g., antibodies or serum proteins of interest), additional nucleic acids, or other analytes. For example, genes and proteins whose expression level/pattern is modulated during the progression of diabetes can be evaluated (e.g., from a biopsy sample, blood sample, urine sample, etc. from the subject). In certain embodiments, additional analytes may be used to evaluate additional characteristics, including but not limited to: microalbumin levels in urine samples for determining microalbuminuria; biomarkers of incipient nephropathy; age or body mass index associated genes; immune tolerance markers; genes found in literature surveys with immune modulatory roles. In addition, other function-related genes may be evaluated, e.g., for assessing sample quality, sampling error in biopsy-based studies, cell surface markers, and normalizing antibodies for calibrating results.

The antibody signature of a sample can be obtained using any convenient method for antibody or protein/peptide analysis. As such, no limitation in this regard is intended. Exemplary peptide analysis includes, but is not limited to: HPLC, mass spectrometry, LC-MS based peptide profiling (e.g., LC-MALDI), Multiple Reaction Monitoring (MRM), ELISA, protein microarray profiling, and the like.

In practicing the methods of the present invention, any convenient protein evaluation/quantitation protocol may be employed, where the levels of one or more autoantibodies/proteins in the assayed sample are determined to generate an antibody signature for the sample. Representative methods include, but are not limited to: MRM analysis, standard immunoassays (e.g., ELISA assays, Western blots, FACS based protein analysis, etc.), protein activity assays, proteomic array assays, flow cytometry, mass spectrometry, etc. For example, antibody levels may be determined by readily adapting methods that are described by Robinson et al. (“Protein arrays for autoantibody profiling and fine-specificity mapping.” Proteomics 2003, 3, 2077-2084) and Quintana et al. (“Functional immunomics: microarray analysis of IgG autoantibody repertoires predicts the future response of mine to induced diabetes”, PNAS, 2004, 101, 14615-14621), the disclosure of which is incorporated by reference in its entirety.

In some instances, protein arrays/microarrays are employed. The terms “array” and “microarray” are used interchangeably herein. A protein array may include one or more known polypeptides (antigens) immobilized at known locations on a solid support. The arrayed polypeptides are potentially capable of capturing an antibody from the subject sample. A protein array may include 10 or more, 25 or more, 50 or more, 100 or more, or 1000 or more, including 5000 or more, 10,000 or more, or 20,000 or more different proteins. A protein array employed in methods of the invention can be constructed anew or may be commercially available, e.g. ProtoArray® Human Protein Microarrays (Invitrogen).

In practicing certain embodiments the methods where arrays are employed, once the array is contacted with a sample, the antibodies from the sample bind to their respective target antigens on the protein array. The array may be subjected to one or more washes as desired, e.g., to remove excess sample, including unbound constituents. Next, a detection step is employed. Detection methods depend on how the samples were prepared prior to contact with the array, but may, for example, include direct or indirect immunofluorescence, as well as colorimetric techniques based on silver-precipitation, chemiluminescence, label free Surface Plasmon Resonance, etc. In some instances, detection may include indirect immunofluorescence, in which the array, following the wash steps described above, is contacted with a secondary antibody (directed against the species from which the sample was derived, e.g. anti-human) that is conjugated to a fluorescent molecule, i.e. a fluorophore, The array is then scanned, using any one of a number of microarray scanners that are standard in the art, to produce an image. The spots on the resulting image can be quantified by commonly used microarray quantification software packages. The resulting location and intensity of each spot can be used to determine the identity and quantity of the antibodies (autoantibodies) that were present in the original sample. Thus, a protein array may be employed to provide the antibody signature from a subject.

Following obtainment of the antibody signature from a subject, the antibody signature is analyzed/evaluated to determine whether the subject has a diabetes phenotype (e.g., whether the subject has diabetes or a progression of diabetes over time with development of ESRD). In certain embodiments, analysis includes comparing the antibody signature with a reference or control signature, e.g., a reference or control; antibody signature, to determine the diabetes phenotype, if any, of the subject. The terms “reference” and “control” as used herein mean a standardized analyte level (or pattern) that can be used to interpret the analyte pattern of a sample from a subject. For example, a reference profile can include autoantibody or target protein level data relating to one or more autoantibodies of interest being evaluated in the sample of the subject/patient. The reference or control profile may be a profile that is obtained from a subject (a control subject) having a diabetes phenotype, and therefore may be a positive reference or control signature for diabetes (e.g., T1D or T2D) or a diabetic renal disease (ESRD). In addition, the reference/control profile may be from a control subject known to not have diabetes and/or ESRD, and therefore be a negative reference/control signature.

In certain embodiments, the obtained antibody signature is compared to a single reference/control profile to determine the subject's diabetes phenotype, if any. In yet other embodiments, the obtained antibody signature is compared to two or more different reference/control profiles to obtain additional or more in depth information regarding the diabetes phenotype, if any, of the subject. For example, the obtained antibody signature may be compared to a positive and negative reference profile to obtain confirmed information regarding the progression or type of diabetes in the subject.

The comparison of the obtained antibody signature and the one or more reference/control profiles may be performed using any convenient methodology, where a variety of methodologies are known to those of skill in the array art, e.g., by comparing digital images of the antibody/protein signatures by comparing databases of peptide signatures and/or gene antibody profiles, etc. Patents describing ways of comparing antibody profiles include, but are not limited to, U.S. Pat. Nos. 6,308,170 and 6,228,575, the disclosures of which are herein incorporated by reference, and may be readily adapted for use in the subject methods.

The comparison step results in information regarding how similar or dissimilar the obtained antibody signature is to the control/reference profile(s), which similarity/dissimilarity information is employed to determine the diabetes phenotype, if any, of the subject. For example, similarity of the obtained antibody signature with the antibody signature of a control sample from a subject experiencing diabetes or a diabetic renal disease indicates that the subject is experiencing diabetes or a diabetic renal disease. Likewise, similarity of the obtained antibody signature with the antibody signature of a control sample from a subject that has not had (or isn't experiencing) diabetes indicates that the subject is not experiencing diabetes.

Depending on the type and nature of the reference/control profile(s) to which the obtained antibody signature is compared, the above comparison step yields a variety of different types of information regarding the subject as well as the sample employed for the assay. As such, the above comparison step can yield a positive/negative determination of an ongoing condition. In certain embodiments, the determination/prediction of diabetes or ESRD can be coupled with a determination of additional characteristics, such as microalbuminuria or incipient nephropathy.

In certain embodiments, a reference profile is a composite reference profile, having control data derived from more than one subject and/or sample. For example, a reference profile may include average autoantibody level data from samples of subjects having experienced the same or similar progression of diabetes.

The subject methods further find use in pharmacological applications. In these applications, a subject/host/patient is first diagnosed with diabetes according to the subject invention, and then treated using a protocol determined, at least in part, on the results of monitoring the diabetes in the subject. For example, a subject may be evaluated for the presence or absence of T1D or T2D using a protocol such as the diagnostic protocol described above. If T1D or T2D is present, the subject may be monitored using a method described herein to determine whether the subject is developing a diabetic kidney disease (e.g., ESRD). The subject may then be treated using a protocol whose suitability is determined using the results of the diagnosing and/or monitoring steps. For example, where the subject is categorized as having a particular diabetes phenotype, therapy can be modulated, e.g., increased or drugs changed, as is known in the art for the treatment/prevention of diabetes and ESRD.

In practicing the subject methods, a subject is typically monitored for diabetes and/or ESRD following receipt of treatment for the same. The subject may be screened once or serially following treatment, e.g., daily, weekly, monthly, bimonthly, half-yearly, yearly, etc. In certain embodiments, the subject is monitored prior to the occurrence of diabetes and/or diabetic kidney disease. In certain other embodiments, the subject is monitored following the occurrence of diabetes and/or diabetic kidney disease.

The subject methods may be employed with a variety of different types of subjects. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) are humans.

Following obtainment of the antibody profile from the sample being assayed, the antibody profile is compared with a reference or control profile to determine the particular diabetes/non-diabetes phenotype of the fluid, cell or tissue, and therefore host, from which the sample was obtained/derived. The terms “reference” and “control” as used herein mean a standardized pattern of levels of autoantibodies to certain proteins to be used to interpret the antibody signature of a given patient and assign a diabetes/non-diabetes phenotype thereto. The reference or control profile may be a profile that is obtained from a fluid/cell/tissue known to have a particular phenotype, e.g., a diabetes phenotype, and therefore may be a positive reference or control profile. In addition, the reference/control profile may be from a fluid/cell/tissue known to not have the phenotype, e.g., a non-diabetes phenotype, and therefore be a negative reference/control profile.

In certain embodiments, the obtained antibody profile is compared to a single reference/control profile to obtain information regarding the phenotype of the fluid/cell/tissue being assayed. In yet other embodiments, the obtained antibody profile is compared to two or more different reference/control profiles to obtain more in depth information regarding the phenotype of the assayed fluid/cell/tissue. For example, the obtained antibody profile may be compared to a positive and negative reference profile to obtain confirmed information regarding whether the fluid/cell/tissue has the phenotype of interest.

The comparison of the obtained antibody profile and the one or more reference/control profiles may be performed using any convenient methodology, e.g., by comparing digital images of the antibody profiles, by comparing databases of expression data, etc. Patents describing ways of comparing antibody profiles include, but are not limited to, U.S. Pat. Nos. 6,308,170 and 6,228,575, the disclosures of which are herein incorporated by reference. Methods of comparing antibody profiles are also described herein, and in the Examples section.

The comparison step results in information regarding how similar or dissimilar the obtained antibody profile is to the control/reference profile(s), which similarity/dissimilarity information is employed to determine the phenotype of the fluid/cell/tissue being assayed. For example, similarity with a positive control indicates that the assayed cell/tissue has a diabetes phenotype. Likewise, similarity with a negative control indicates that the assayed fluid/cell/tissue has a non-diabetes phenotype.

Depending on the type and nature of the reference/control profile(s) to which the obtained antibody profile is compared, the above comparison step yields a variety of different types of information regarding the fluid/cell/tissue that is assayed. As such, the above comparison step can yield a positive/negative determination of a particular phenotype of an assayed fluid/cell/tissue. In many embodiments, the above-obtained information about the fluid/cell/tissue being assayed is employed to diagnose a host, subject or patient with respect to whether that host has diabetes (e.g., T1D or T2D) or is a host with diabetes that has or will develop ESRD in the future, as described above.

The subject methods further find use in pharmacogenomic applications. In these applications, a subject/host/patient is first diagnosed for the presence or absence of the diabetes phenotype using a protocol such as the diagnostic protocol described in the preceding section. The subject is then treated using a protocol whose suitability is determined using the results of the diagnosis step. Where a patient is identified as having a particular diabetes phenotype related to ESRD, suitable therapies and protocols for chronic kidney disease may be employed.

In some embodiments, following diagnosis of diabetes (T1D or T2D) a host is screened for the presence of a diabetes phenotype for ESRD. The host may be screened once or serially following an initial treatment, e.g., daily, weekly, monthly, bimonthly, half-yearly, yearly, etc. In certain embodiments, monitoring of the host antibody profile even after therapy has been reduced or discontinued is conducted to determine whether the host has maintained the antibody profile and may continue for the lifetime of the host.

### Databases of Profiles of Phenotype Determinative Antibodies

Also provided are databases of antibody profiles of diabetes phenotype determinative genes. Such databases will typically comprise antibody profiles of various fluids/cells/tissues having diabetes phenotypes, negative antibody profiles, etc., where such profiles are further described below.

The antibody profiles and databases thereof may be provided in a variety of media to facilitate their use. “Media” refers to a manufacture that contains the antibody profile information of the present invention. The databases of the present invention can be recorded on computer readable media, e.g. any medium that can be read and accessed directly by a user employing a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. Thus, the subject antibody profile databases are accessible by a user, i.e., the database files are saved in a user-readable format (e.g., a computer readable format, where a user controls the computer).

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention, e.g., to and from a user via a graphical user interface. One format for an output means ranks antibody profiles possessing varying degrees of similarity to a reference antibody profile. Such presentation provides a skilled artisan with a ranking of similarities and identifies the degree of similarity contained in the test antibody profile. Embodiments of the subject systems include the following components: (a) a communications module for facilitating information transfer between the system and one or more users, e.g., via a graphical user interface; and (b) a processing module for performing one or more tasks involved in the analysis methods of the invention.

### Reagents, Systems and Kits

Also provided are reagents, systems and kits thereof for practicing one or more of the above-described methods. The subject reagents, systems and kits thereof may vary greatly. Reagents of interest include reagents specifically designed for use in production of the above-described antibody signatures. These include a protein level evaluation element made up of one or more reagents. The term system refers to a collection of reagents, however compiled, e.g., by purchasing the collection of reagents from the same or different sources. The term kit refers to a collection of reagents provided, e.g., sold, together.

The subject systems and kits include reagents for peptide or protein (e.g., autoantibody) level determination, for example those that find use in ELISA assays, Western blot assays, MS assays (e.g., LC-MS), HPLC assays, flow cytometry assays, array based assays, and the like. One type of such reagent is one or more probe specific for one or more autoantibodies to proteins listed in Tables 1-4. For example, the target proteins of Tables 1-4 or fragments thereof (as are well known in the art) find use in the subject systems as probes. In certain embodiments, protein arrays containing target proteins at known locations on a substrate are provided in the subject systems (see, e.g., U.S. Pat. Nos.: 4,591,570; 5,143,854; 7,354,721; the disclosures of which are herein incorporated by reference, and may be readily adapted for use in the embodiments described herein). Probes for any combination of autoantibodies described herein may be employed. The subject arrays may include probes for one or more autoantibodies to only those proteins that are listed in Tables 1-4 or may include additional probes that are not listed therein, such as probes for proteins whose level can be used to evaluate additional characteristics as well as other array assay function related proteins, e.g., for assessing sample quality, sampling error, and normalizing protein levels for calibrating results, and the like.

The systems and kits of the subject invention may include the above-described arrays and/or specific probes or probe collections. The systems and kits may further include one or more additional reagents employed in the various methods, such as various buffer mediums, e.g. incubation and washing buffers, prefabricated probe arrays, labeled probe purification reagents and components, like spin columns, etc., signal generation and detection reagents, e.g. secondary antibodies (e.g., conjugated to detectable moieties, e.g., horseradish peroxidase (HRP), alkaline phosphatase, etc.), chemifluorescent or chemiluminescent substrates, fluorescent moieties, and the like.

The subject systems and kits may also include a phenotype determination element, which element is, in many embodiments, a reference or control protein/peptide (e.g., antibody) signature or gene expression profile that can be employed, e.g., by a suitable computing means, to determine a diabetes phenotype based on an “input” antibody signature. Representative phenotype determination elements include databases of antibody signatures, e.g., reference or control profiles, as described above.

In addition to the above components, the subject systems/kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Aspects of the present invention thus provide systems for diagnosing and monitoring diabetes in a subject. The system includes: an autoantibody level evaluation element configured for evaluating the level of one or more autoantibodies in a sample from a subject to obtain an antibody signature, where the one or more autoantibodies includes an antibody to a protein of Tables 1-4; and a phenotype determination element configured for employing the antibody signature to determine whether the subject has a diabetes phenotype.

The following examples are offered by way of illustration and not by way of limitation.

### Experimental

Methods

Discovery Method

Sera collected from Type-1 diabetes (T1D) patients, Type-2 diabetes (T2D) patients, End Stage Renal Disease (ESRD) patients, and healthy patients was analyzed by protein microarray. High-density protein-arrays, ProtoArray® Human Protein Microarray V5, with approximately 9400 highly purified full-length human proteins were used to profile IgG antibodies from different phenotypes of diabetes. Microarray slides were blocked in blocking buffer (50 mM HEPES, 200 mM NaCl, 0.08% Triton X-100, 25% glycerol, 20 mM reduced glutathione, 1.0 mM DTT, 1% Hammarsten Grade casein) at 4° C. for 1 hour. After blocking, arrays were removed from the blocking solution and probed with a 1:500 dilution of each serum sample diluted in 5 mL of freshly prepared PBST buffer (1× PBS, 0.1% Tween 20, 1% Hammarsten Grade casein) on lot-matched ProtoArray® Protein Microarrays. Arrays were then incubated for 90 minutes at 4° C. in QuadriPERM 4-well trays (Greiner) with gentle agitation. After incubation, slides were washed five times (5 minutes per wash) in 5 ml PBST Buffer in 4-well trays. An Alexa Fluor®647-conjugated goat anti-human IgG Ab diluted in 5 ml probe buffer to a 1 μg/ml final concentration were added to each array and were incubated with gentle shaking at 4° C. for 90 minutes. After incubation, the secondary Ab was removed, and arrays were washed as described above. Arrays were dried by spinning in a table-top centrifuge equipped with a plate rotor at 1000 rpm for 2 minutes. Arrays were then scanned using an Axon GenePix 4000B fluorescent microarray scanner. Data was analyzed by using Prospector Analyzer® (Invitrogen). A list of significantly increased antibodies in each phenotypes were identified in terms of fold increase and p value Wilcoxon (rank sum) test.

### Example 1

AutoAbs were identified against a number of human antigens, the reactivities of which were significantly increased in T1 D sera compared to sera from healthy normal controls (Table 1). The list includes autoABs specific for aspartoacylase (aminocyclase) 3 (ACY3), AMME chromosomal region gene 1-like (AMMECR1L), basic leucine zipper transcription factor, ATF-like 2 (BATF2), BMX non-receptor tyrosine kinase (BMX), EPH receptor A2 (EPHA2), fms-related tyrosine kinase 1 (vascular endothelial growth factor/vascular permeability factor receptor) (FLT1), p21 protein (Cdc42/Rac)-activated kinase 4 (PAK4), TRAF3 interacting protein 2 (TRAF3IP2). Also included are chromosome 9 open reading frame 25 (C9ORF25), chromosome X open reading frame 38 (CXORF38), and chromosome X open reading frame 56 (CXORF56).

TABLE 1
Antibody biomarkers for Type 1 Diabetes

| S. | | |
|---|---|---|
| No. | Gene Symbol | Protein Name |
| 1 | SNRPB2 | small nuclear ribonucleoprotein polypeptide |
| | | B″ (SNRPB2), transcript variant 1 |
| 2 | DDX42 | DEAD (Asp-Glu-Ala-Asp) box polypeptide |
| | | 42 (DDX42) |
| 3 | C11orf63 | chromosome 11 open reading frame 63 |
| | | (C11orf63), transcript variant 2 |
| 4 | TCOF1 | Treacher Collins-Franceschetti syndrome |
| | | 1 (TCOF1), transcript variant 3 |
| 5 | TSSK2 | testis-specific serine kinase 2 (TSSK2) |
| 6 | KDM4B | JmjC domain-containing histone |
| | | demethylation protein 3B |
| 7 | PDGFB | platelet-derived growth factor beta polypeptide |
| | | (simian sarcoma viral (v-sis) oncogene |
| | | homolog) (PDGFB), transcript variant 1 |
| 8 | LTK | Leukocyte tyrosine kinase receptor |
| 9 | RPL14 | ribosomal protein L14 (RPL14) |
| 10 | VIM | Vimentin |
| 11 | GTF2I | General transcription factor II-I |
| 12 | BCL2L13 | BCL2-like 13 (apoptosis facilitator) |
| | | (BCL2L13) |
| 13 | LARP6 | La ribonucleoprotein domain family, |
| | | member 6 (LARP6), transcript variant 1 |
| 14 | DKFZP434K028 | DKFZP434K028 protein (DKFZP434K028) |
| 15 | USP39 | ubiquitin specific peptidase 39 (USP39) |
| 16 | SERBP1 | SERPINE1 mRNA binding protein 1 |
| | | (SERBP1) |
| 17 | CCL19 | chemokine (C-C motif) ligand 19 (CCL19) |
| 18 | GAD2 | Glutamate decarboxylase 2 |
| 19 | MCM10 | cDNA clone IMAGE: 3451214 (MCM10) |
| 20 | ZNF688 | zinc finger protein 688 (ZNF688), transcript |
| | | variant 1 |
| 21 | PTEN | Phosphatidylinositol-3,4,5-trisphosphate |
| | | 3-phosphatase and dual-specificity protein |
| | | phosphatase PTEN |
| 22 | RP6-166C19.11 | cancer/testis CT47 family, member 11 |
| | | (CT47.11) |
| 23 | GIPC1 | PDZ domain-containing protein GIPC1 |
| 24 | TIGD1 | Tigger transposable element-derived protein 1 |
| 25 | CCDC131 | coiled-coil domain containing 131 (CCDC131) |
| 26 | HTF9C | HpaII tiny fragments locus 9c protein |
| 27 | SOX5 | SRY (sex determining region Y)-box 5 |
| | | (SOX5), transcript variant 3 |
| 28 | MCF2L | Guanine nucleotide exchange factor DBS |
| 29 | TRAF3IP1 | TRAF3-interacting protein 1 |
| 30 | 6CKINE | C-C motif chemokine 21 |
| 31 | ACY3 | aspartoacylase (aminocyclase) 3 |
| 32 | AMMECR1L | AMME chromosomal region gene 1-like |
| 33 | ARHGAP9 | Rho GTPase activating protein 9 |
| 34 | ASNS | asparagine synthetase |
| 35 | BATF2 | basic leucine zipper transcription factor, |
| | | ATF-like 2 |
| 36 | BMX | BMX non-receptor tyrosine kinase |
| 37 | C9ORF25 | chromosome 9 open reading frame 25 |
| 38 | CDC2 | cell division cycle 2, G1 to S and G2 to M |
| 39 | CHGB | chromogranin B (secretogranin 1) |
| 40 | CXORF38 | chromosome X open reading frame 38 |
| 41 | CXORF56 | chromosome X open reading frame 56 |
| 42 | DMD | dystrophin |
| 43 | ECHDC1 | enoyl Coenzyme A hydratase domain |
| | | containing 1 |
| 44 | EIF3F | eukaryotic translation initiation factor 3, |
| | | subunit F |
| 45 | EPHA2 | EPH receptor A2 |
| 46 | ERMN | ermin, ERM-like protein |
| 47 | FAM136A | family with sequence similarity 136, member A |
| | (includes | |
| | EG: 84908) | |
| 48 | FILIP1 | filamin A interacting protein 1 |
| 49 | FLT1 | fms-related tyrosine kinase 1 (vascular |
| | | endothelial growth factor/vascular permeability |
| | | factor receptor) |
| 50 | GART | phosphoribosylglycinamide formyltransferase, |
| | | phosphoribosylglycinamide synthetase, |
| | | phosphoribosylaminoimidazole synthetase |
| 51 | GIMAP6 | GTPase, IMAP family member 6 |
| 52 | GNG7 | guanine nucleotide binding protein |
| | | (G protein), gamma 7 |
| 53 | GTF2F1 | general transcription factor IIF, |
| | | polypeptide 1, 74 kDa |
| 54 | HGS | hepatocyte growth factor-regulated tyrosine |
| | | kinase substrate |
| 55 | IFI6 | interferon, alpha-inducible protein 6 |
| 56 | KDM4B | lysine (K)-specific demethylase 4B |
| 57 | LACE1 | lactation elevated 1 |
| 58 | LGALS1 | lectin, galactoside-binding, soluble, 1 |
| 59 | LGALS7 | lectin, galactoside-binding, soluble, 7 |
| 60 | LIMS2 | LIM and senescent cell antigen-like domains 2 |
| 61 | LTK | leukocyte receptor tyrosine kinase |
| 62 | LUC7L | LUC7-like (S. cerevisiae) |
| 63 | NCAPG | non-SMC condensin I complex, subunit G |
| | (includes | |
| | EG: 64151) | |
| 64 | NME6 | non-metastatic cells 6, protein expressed |
| | | in (nucleoside-diphosphate kinase) |
| 65 | NUPL1 | nucleoporin like 1 |
| 66 | PAK4 | p21 protein (Cdc42/Rac)-activated kinase 4 |
| 67 | PDE4DIP | phosphodiesterase 4D interacting protein |
| 68 | PSIP1 | PC4 and SFRS1 interacting protein 1 |
| 69 | RAB20 | RAB20, member RAS oncogene family |
| 70 | RNGTT | RNA guanylyltransferase and 5′-phosphatase |
| 71 | RPS3 | ribosomal protein S3 |
| 72 | SPG20 | spastic paraplegia 20 (Troyer syndrome) |
| 73 | TALDO1 | transaldolase 1 |
| 74 | TBRG1 | transforming growth factor beta regulator 1 |
| 75 | THAP1 | THAP domain containing, apoptosis associated |
| | | protein 1 |
| 76 | TRAF3IP2 | TRAF3 interacting protein 2 |
| 77 | UBL4A | ubiquitin-like 4A |
| 78 | ZC3HC1 | zinc finger, C3HC-type containing 1 |
| 79 | ZNF131 | zinc finger protein 131 |

### Example 2

AutoAbs were identified against a number of human antigens, the reactivities of which were significantly increased in T2D sera compared to sera from patients with other kidney diseases (ESRD) (Table 2). The list includes autoABs specific for

TABLE 2
Antibody biomarkers for Type 2 Diabetes over
other kidney diseases (ESRD).

| S. No. | Gene Symbol | Protein Name |
|---|---|---|
| 1 | RAD51AP1 | RAD51 associated protein 1 (RAD51AP1) |
| 2 | HADH | hydroxyacyl-Coenzyme A dehydrogenase |
| | (HADH) | |
| 3 | C11orf16 | chromosome 11 open reading frame 16 |
| | (C11orf16) | |
| 4 | TAC3 | Tachykinin-3 |
| 5 | ABR | active BCR-related gene (ABR), transcript |
| | | variant 2 |
| 6 | ECE1 | endothelin converting enzyme 1 (ECE1) |
| 7 | PPP1R2 | protein phosphatase 1, regulatory (inhibitor) |
| | | subunit 2 (PPP1R2) |
| 8 | GRINL1A | glutamate receptor, ionotropic, N-methyl |
| | | D-aspartate-like 1A (GRINL1A), transcript |
| | | variant 1 |
| 9 | ABR | active BCR-related gene (ABR), transcript |
| | | variant 1 |
| 10 | C19orf44 | Uncharacterized protein C19orf44 |
| 11 | MUSTN1 | musculoskeletal, embryonic nuclear protein 1 |
| | | (MUSTN1) |
| 12 | ETHE1 | ethylmalonic encephalopathy 1 (ETHE1) |
| 13 | BMI1 | BMI1 polycomb ring finger oncogene (BMI1) |
| 14 | BAZ2B | bromodomain adjacent to zinc finger domain, |
| | | 2B, I mRNA (cDNA clone MAGE: 4290975), |
| | | complete cds. |
| 15 | TBC1D22A | TBC1 domain family, member 22A |
| | | (TBC1D22A) |
| 16 | CAMK2N2 | calcium/calmodulin-dependent protein |
| | | kinase II inhibitor 2 (CAMK2N2) |
| 17 | ASS1 | argininosuccinate synthetase 1 (ASS1), |
| | | transcript variant 2 |
| 18 | CCNY | Cyclin-Y |
| 19 | MARK2 | MAP/microtubule affinity-regulating kinase 2 |
| | | (MARK2), transcript variant 3 |
| 20 | RAD51AP1 | RAD51 associated protein 1 (RAD51AP1) |
| 21 | RAB38 | RAB38, member RAS oncogene family |
| | | (RAB38) |
| 22 | RIOK1 | RIO kinase 1 (yeast) (RIOK1) |
| 23 | HSP90AA1 | Heat shock protein HSP 90-alpha |
| 24 | C11orf74 | chromosome 11 open reading frame 74 |
| | | (C11orf74) |
| 25 | ARID3A | AT rich interactive domain 3A (BRIGHT-like) |
| | | (ARID3A) |
| 26 | LMOD1 | Leiomodin-1 |
| 27 | CAPRIN1 | cell cycle associated protein 1 (CAPRIN1), |
| | | transcript variant 1 |
| 28 | ITGB3BP | Centromere protein R |
| 29 | MND1 | Meiotic nuclear division protein 1 homolog |
| 30 | SGK | serum/glucocorticoid regulated kinase (SGK) |

### Example 3

AutoAbs were identified against a number of human antigens, the reactivities of which were significantly increased in T2D sera compared to sera from healthy normal controls (Table 3). The list includes autoABs specific for

TABLE 3
Antibody Biomarkers for Type 2 Diabetes over healthy controls

| S. No. | Gene Symbol | Protein Name |
|---|---|---|
| 1 | NADK | NAD kinase (NADK) |
| 2 | MED9 | mediator complex subunit 9 (MED9) |
| 3 | LDHA | lactate dehydrogenase A (LDHA) |
| 4 | ARHGAP26 | Rho GTPase activating protein 26 (ARHGAP26) |
| 5 | ANKRA2 | ankyrin repeat, family A (RFXANK-like), 2 (ANKRA2) |
| 6 | CRY2 | cryptochrome 2 (photolyase-like) (CRY2) |
| 7 | IL23A | interleukin 23, alpha subunit p19 (IL23A) |
| 8 | DUSP14 | dual specificity phosphatase 14 (DUSP14) |
| 9 | ZBTB44 | zinc finger and BTB domain containing 44 (ZBTB44) |
| 10 | SIRT1 | NAD-dependent deacetylase sirtuin-1 |
| 11 | SLC2A3 | solute carrier family 2 (facilitated glucose transporter), member 3 |
| | | (SLC2A3) |
| 12 | GPR172B | G protein-coupled receptor 172B (GPR172B), transcript variant 2 |
| 13 | CCDC89 | coiled-coil domain containing 89 (CCDC89) |
| 14 | BATF | basic leucine zipper transcription factor, ATF-like (BATF) |
| 15 | HMOX1 | Heme oxygenase 1 |
| 16 | ARRDC1 | arrestin domain containing 1 (ARRDC1) |
| 17 | USF2 | Upstream stimulatory factor 2 |
| 18 | GBGT1 | globoside alpha-1,3-N-acetylgalactosaminyltransferase 1 (GBGT1) |
| 19 | EDC3 | enhancer of mRNA decapping 3 homolog (S. cerevisiae) (EDC3) |
| 20 | SGIP1 | SH3-domain GRB2-like (endophilin) interacting protein 1 (SGIP1) |
| 21 | GCGR | glucagon receptor (GCGR) |
| 22 | ZRANB2 | zinc finger, RAN-binding domain containing 2 (ZRANB2) |
| 23 | NLGN4Y | neuroligin 4, Y-linked (NLGN4Y) |
| 24 | GJB6 | gap junction protein, beta 6 (GJB6) |
| 25 | CDK10 | cyclin-dependent kinase (CDC2-like) 10 (CDK10), transcript variant a |
| 26 | PSG1 | Pregnancy-specific beta-1-glycoprotein 1 |
| 27 | CCDC74A | Coiled-coil domain-containing protein 74A |
| 28 | DENND1C | DENN/MADD domain containing 1C (DENND1C) |
| 29 | MAP2K6 | mitogen-activated protein kinase kinase 6 (MAP2K6), transcript variant |
| | | 2; see catalog number for detailed information on wild-type or point |
| | | mutant status |

### Example 4

AutoAbs were identified against a number of human antigens, the reactivities of which were significantly increased in End Stage Renal Disease (ESRD) sera compared to sera from healthy normal controls (Table 4). The list includes autoABs specific for

TABLE 4
Antibody Biomarkers for End Stage Renal Disease (ESRD)

| S. No. | Gene Symbol | Protein Name |
|---|---|---|
| 1 | IGLC1 | immunoglobulin lambda constant 1 (Mcg marker) (IGLC1) |
| 2 | IGHG1 | Ig gamma-1 chain C region |
| 3 | EDC3 | enhancer of mRNA decapping 3 homolog (S. cerevisiae) (EDC3) |
| 4 | IGK@ | cDNA clone MGC: 22645 IMAGE: 4700961, complete cds |
| 5 | IGHG1 | Ig gamma-1 chain C region |
| 6 | APEX2 | APEX nuclease (apurinic/apyrimidinic endonuclease) 2 (APEX2), nuclear |
| | | gene encoding mitochondrial protein |
| 7 | CD3D | cDNA clone MGC: 27152 IMAGE: 4691630, complete cds |
| 8 | TRIM21 | tripartite motif-containing 21 (TRIM21) |
| 9 | IGK@ | cDNA clone MGC: 27376 IMAGE: 4688477, complete cds |
| 10 | IGKV1-5 | immunoglobulin kappa variable 1-5 (IGKV1-5) |
| 11 | IGHG3 | immunoglobulin heavy constant gamma 3 (G3m marker) (IGHG3) |
| 12 | CTLA-FC | Recombinant human CTLA-4/Fc |
| 13 | IGL@ | immunoglobulin lambda locus (IGL@) |
| 14 | CD7 | cDNA clone MGC: 32654 IMAGE: 4701898, complete cds |
| 15 | CLIP4 | CAP-GLY domain containing linker protein family, member 4 (CLIP4) |
| 16 | MAPRE1 | microtubule-associated protein, RP/EB family, member 1 (MAPRE1) |
| 17 | SNRPB2 | small nuclear ribonucleoprotein polypeptide B″ (SNRPB2), transcript variant 1 |
| 18 | IGHG1 | Ig gamma-1 chain C region |
| 19 | ZBTB44 | zinc finger and BTB domain containing 44 (ZBTB44) |
| 20 | CD3D | Ig lambda chain C regions |
| 21 | IGHG1 | immunoglobulin heavy constant gamma 1 (G1m marker) (IGHG1) |
| 22 | TRAM1 | translocation associated membrane protein 1 (TRAM1) |
| 23 | ERR beta- | Estrogen Related Receptor beta, Ligand Binding Domain (ERR beta-LBD) |
| | LBD | |
| 24 | CNBP | CCHC-type zinc finger, nucleic acid binding protein (CNBP) |
| 25 | N/A | cDNA clone MGC: 18299 IMAGE: 4179890, complete cds |
| 26 | OLFM1 | olfactomedin 1 (OLFM1), transcript variant 1 |
| 27 | IGHM | immunoglobulin heavy constant mu (IGHM) |
| 28 | SIRT5 | sirtuin (silent mating type information regulation 2 homolog) 5 (S. cerevisiae) |
| | | (SIRT5), transcript variant 1 |
| 29 | CEP290 | centrosomal protein 290 kDa (CEP290) |
| 30 | PHLDA1 | pleckstrin homology-like domain, family A, member 1 (PHLDA1) |

### Conclusion

Diabetes (T1D and T2D) and End Stage Renal Disease (ESRD) are associated with elevated IgG autoantibodies that recognize multiple different proteins. This analysis of the autoantibody signature in diabetes and ESRD identifies protein targets that are of interest in both the diagnosis and monitoring of diabetes in a subject.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for diagnosing or monitoring diabetes in a subject, the method comprising:
evaluating the level of one or more autoantibody in a sample from the subject to obtain an antibody signature, wherein the one or more autoantibody is selected from an antibody to a protein set forth in Tables 1-4; and
determining whether the subject has a diabetes phenotype based on the antibody signature.

2. The method of claim 1, wherein the diabetes phenotype is for type 1 diabetes.

3. The method of claim 1, wherein the diabetes phenotype is for type 2 diabetes.

4. The method of claim 1, wherein the subject has diabetes and the diabetes phenotype is for ESRD.

5. The method of claim 2, wherein the level of 5 or more autoantibodies to proteins from Table 1 is evaluated.

6. The method of claim 5, wherein the level of 10 or more autoantibodies to proteins from Table 1 is evaluated.

7. The method of claim 6, wherein the level of 15 or more autoantibodies to proteins from Table 1 is evaluated.

8. The method of claim 7, wherein autoantibodies to all of the proteins listed in Table 1 are evaluated.

9. The method of claim 3, wherein the level of 5 or more autoantibodies to proteins from Table 3 is evaluated.

10. The method of claim 9, wherein the level of 10 or more autoantibodies to proteins from Table 3 is evaluated.

11. The method of claim 10, wherein the level of 15 or more autoantibodies to proteins from Table 3 is evaluated.

12. The method of claim 11, wherein autoantibodies to all of the proteins listed in Table 3 are evaluated.

13. The method of claim 4, wherein the level of 5 or more autoantibodies to proteins from Tables 2 and 4 is evaluated.

14. The method of claim 13, wherein the level of 10 or more autoantibodies to proteins from Tables 2 and 4 is evaluated.

15. The method of claim 14, wherein the level of 15 or more autoantibodies to proteins from Tables 2 and 4 is evaluated.

16. The method of claim 15, wherein autoantibodies to all of the proteins listed in Tables 2 and 4 are evaluated.

17. The method of claim 1, wherein the sample is a blood sample.

18. The method of claim 1, wherein said determining step comprises comparing said antibody signature to a reference.

19. The method of claim 1, wherein the subject is determined to have diabetes when the level of the one or more autoantibody in the sample is increased as compared a reference antibody signature.

20. The method of claim 1, wherein the evaluating step comprises a protein microarray assay.

21. A system for diagnosing or monitoring diabetes in a subject, said system comprising:
a protein level evaluation element configured for evaluating the level of one or more autoantibody in a sample from a subject to obtain an antibody signature, wherein the one or more autoantibody is selected from an autoantibody to the proteins of Tables 1-4; and
a phenotype determination element configured for employing the antibody signature to determine whether the subject has a diabetes phenotype.

22. The system according to claim 21, wherein the protein level evaluation element comprises at least one reagent for assaying the level of an autoantibody to a protein listed in Table 1 in the sample.

23. The system according to claim 21, wherein the protein level evaluation element comprises at least one reagent for assaying the level of an autoantibody to a protein listed in Table 3 in the sample.

24. The system according to claim 21, wherein the protein level evaluation element comprises at least one reagent for assaying the level of an antibody to a protein listed in Tables 2 and 4 in the sample.

25. The system according to claim 21, wherein the phenotype determination element comprises one or more reference antibody signatures to which the antibody signature is compared to determine whether the subject has a diabetes phenotype.
